(19) United States
(12) Patent Application Publication
(10) Pub. No.: US 2019/0167492 A1
(43) Pub. Date: Jun. 6, 2019

(54) Travel Diaper

(71) Applicant: Quincy Deward Williams, Chester, VA (US)

(72) Inventor: Quincy Deward Williams, Chester, VA (US)

(21) Appl. No.: 15/860,236

(22) Filed: Jan. 2, 2018

Related U.S. Application Data
(63) Continuation-in-part of application No. 29/628,315, filed on Dec. 4, 2017, now Pat. No. D839420.

(51) Int. Cl. A61F 13/49; A61F 13/551

(52) U.S. Cl. CPC A61F 13/49058; A61F 2013/55125; A61F 13/5519; A61F 13/5512

(57) ABSTRACT

A travel diaper is an apparatus that allows a user easy access to a plurality of wipes and a disposable bag during and after the process of changing a diaper. The travel diaper includes a diaper body, a plurality of wipes, a disposable bag, a first access flap, and a second access flap. The diaper body provides a standard diaper structural body. The plurality of wipes provides cleaning material for an individual during the process of changing a diaper. The first access flap conceals the plurality of wipes and attaches the plurality of wipes to the diaper body. The disposable bag provides a waste receptacle to receive the used travel diaper after the process of the changing a diaper. The second access flap conceals the disposable bag and attaches the disposable bag to the diaper body.

### FIELD OF THE INVENTION

The present invention relates generally to baby care products. More particularly, the present invention is a travel diaper which includes a plurality of wipes and a disposable bag attached to the diaper body through access flaps. The present invention additionally includes a plurality of diaper-changing accessory compartments for the storage of various diaper-changing accessories.

### BACKGROUND OF THE INVENTION

Diapers are very important baby products that allow an individual, primarily infants, to defecate or urinate without the use of a toilet. Diapers are well-known baby care products which have been used for many years for this purpose. Other well-known baby care products are baby wipes, powders, ointments, etc. to help with skin rashes or skin sensitivity and to clean the wearer of the diaper. The baby care products are typically stored in and obtained from family homes, nurseries, and healthcare facilities. When the individual has been changed to a new diaper, the old diaper is typically disposed of into any bag then into a waste receptacle. Unfortunately, any of the listed places or similar and baby care products may not always be nearby and therefore, an individual may not have access to the baby care products and/or a bag to dispose of the diaper. For example, an individual may forget to carry the baby care products needed for the changing of a diaper.

It is therefore an objective of the present invention to provide a travel diaper which includes a plurality of wipes and a disposable bag attached to the diaper body through access flaps. The plurality of wipes is readily available for the user when changing a diaper by opening the corresponding access flap which attaches the plurality of wipes to the diaper body. Additionally, when the diaper has been changed, the user can access to a disposable bag by opening the corresponding access flap which attaches the disposable bag to the diaper body. The present invention additionally includes a plurality of diaper-changing accessory compartments that allow a user to store his or her own diaper-changing accessories in order for easy access when changing a diaper.

### BRIEF DESCRIPTION OF THE DRAWINGS

FIG. 1 is a rear-top perspective view of the present invention.

FIG. 2 is a rear-bottom perspective view of the present invention.

FIG. 3 is a front-bottom perspective view of the present invention.

FIG. 4 is a schematic diagram of the present invention displaying the plurality of wipes and the disposable bag.

FIG. 5 is a rear-top perspective view of an alternative embodiment of the present invention.

### DETAIL DESCRIPTIONS OF THE INVENTION

All illustrations of the drawings are for the purpose of describing selected versions of the present invention and are not intended to limit the scope of the present invention.

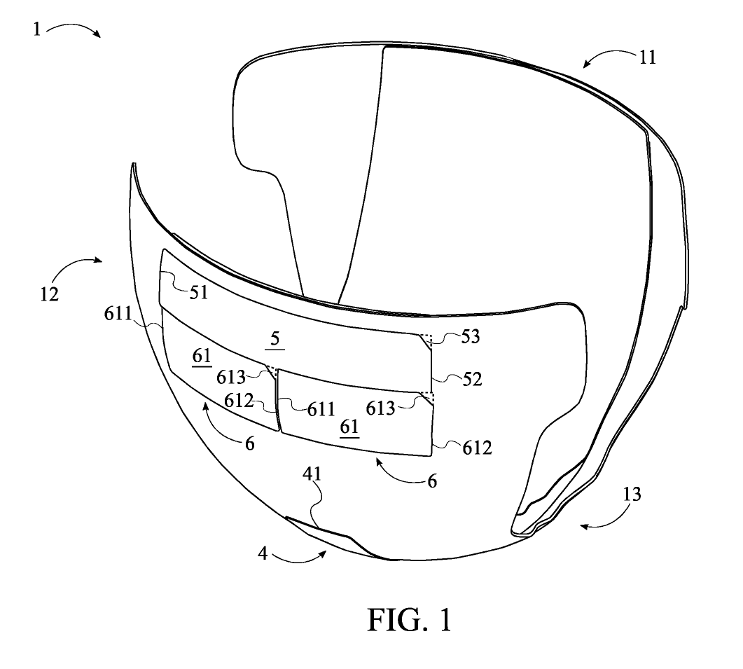

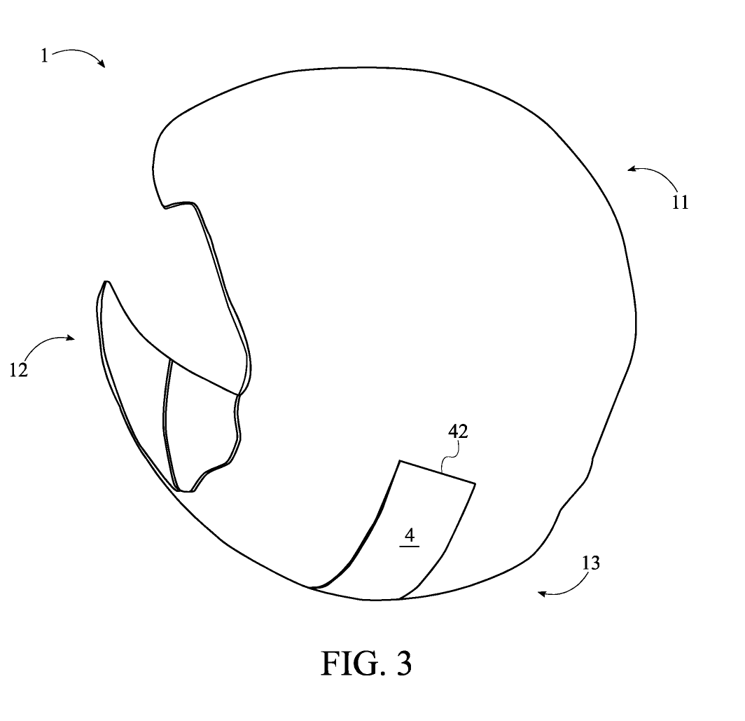

In reference to FIGS. 1 through 5, the present invention is a travel diaper which allows a user to readily access cleaning material, a waste receptacle, and diaper-changing accessories during and after the process of changing a diaper. The present invention comprises a diaper body 1, a plurality of wipes 2, a disposable bag 3, a first access flap 4, and a second access flap 5. The diaper body 1 functions as the main structural body within the present invention so that the plurality of wipes 2, the disposable bag 3, the first access flap 4, and the second access flap 5 can be positioned and connected onto the present invention. The plurality of wipes 2 allows the user to clean an individual, primarily an infant, when changing the diaper of said individual. The disposable bag 3 allows the user to dispose of the diaper body 1 after usage. The first access flap 4 conceals and attaches the plurality of wipes 2 to the diaper body 1. The second access flap 5 conceals and attaches the disposable bag 3 to the diaper body 1.

The diaper body 1 has a variety of reference points in order to properly position the diaper body 1 on the present invention. Thus, the diaper body 1 comprises an anterior-bracing portion 11, a posterior-bracing portion 12, and a crotch-bracing portion 13. The anterior-bracing portion 11, the posterior-bracing portion 12, and the crotch-bracing portion 13 form the standard structural shape of a diaper. The diaper body 1 can be composed of any material such as, but not limited to, breathable, stretchable, and/or absorbable material. The diaper body 1 can be any size allowing a range of users, from infants to adults, to utilize the present invention. The diaper body 1 will include a fastening means to secure the present invention around the waist of the wearer.

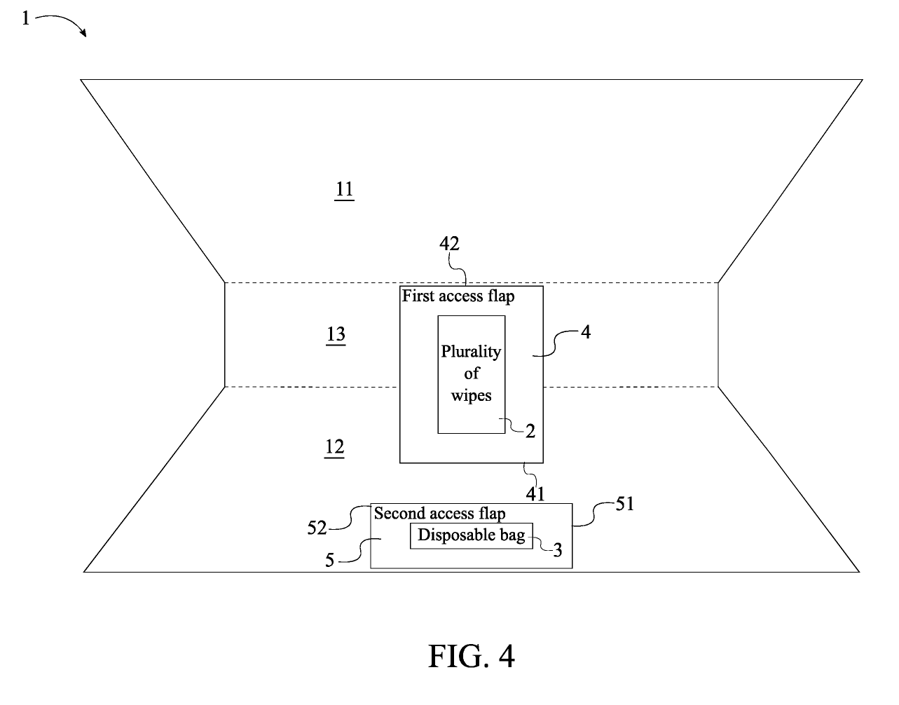

The general configuration of the aforementioned components allows the user to readily access cleaning material, a waste receptacle, and diaper changing accessories during and after changing a diaper. With reference to FIG. 4, the plurality of wipes 2 and the disposable bag 3 is externally positioned to the diaper body 1. More specifically, the external arrangement of the plurality of wipes 2 and the disposable bag 3 with respect to the diaper body 1 allows the plurality of wipes 2 to be easily accessible to the user. Furthermore, the plurality of wipes 2 and the disposable bag 3 are externally positioned to the diaper body 1 in order to not compromise the structure of the diaper body 1 and ensure the maximum comfort and safety for the wearer of the present invention.

The plurality of wipes 2 is removably attached to the diaper body 1 by the first access flap 4. The arrangement between the first access flap 4 and the plurality of wipes 2 allows the plurality of wipes 2 to be concealed and secured to the diaper body 1 while still being easily accessible by opening the first access flap 4. The plurality of wipes 2 is positioned between the diaper body 1 and first access flap 4. The plurality of wipes 2 may be composed of any material which will be comfortable for the skin of the wearer. The plurality of wipes 2 may be scented and/or be lotion-based cleaning wipes.

The disposable bag 3 is removably attached to the diaper body 1 by the second access flap 5. The arrangement between the second access flap 5 and the disposable bag 3 allows the disposable bag 3 to be concealed and secured to the diaper body 1 while still being easily accessible by opening the second access flap 5. The disposable bag 3 is positioned between the diaper body 1 and the second access flap 5. The disposable bag 3 may be composed of any material which is expandable allowing the user to throw out the present invention, inside the disposable bag 3, in any external waste receptacle. The disposable bag 3 may include a sealing means to securely maintain the present invention inside of the disposable bag 3 after the diaper-changing process.

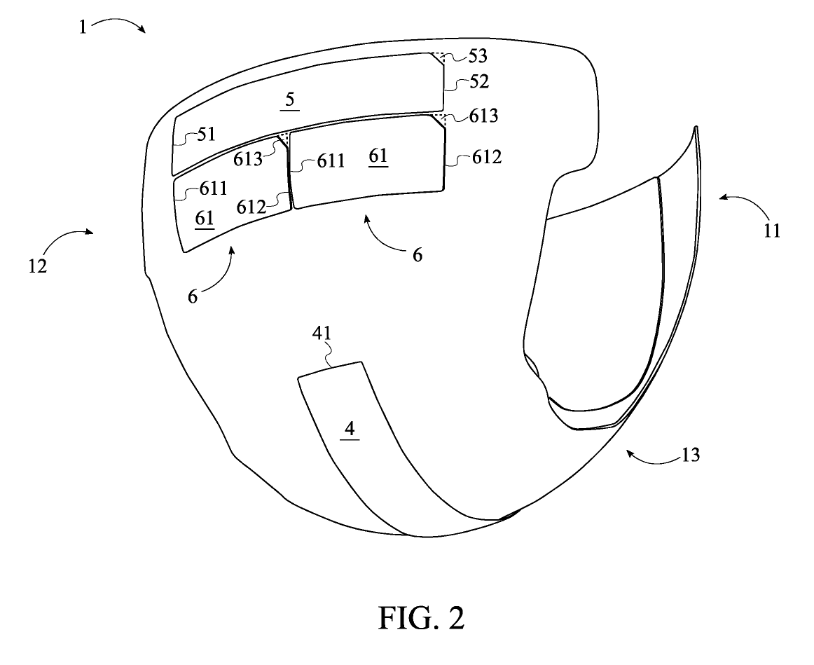

With reference to FIGS. 2 through 4 and in the preferred embodiment of the present invention, the plurality of wipes 2 is positioned onto the crotch-bracing portion 13 and posterior-bracing portion 12 and is positioned adjacent to the anterior-bracing portion 11 giving the user easy access to the plurality of wipes 2 during the diaper-changing process. Furthermore, the positioning of the plurality of wipes 2 does not restrict movement of the wearer while wearing the present invention. Furthermore, the first access flap 4 comprises a first fixed edge 41 and a first free edge 42. The first fixed edge 41 and the first free edge 42 are positioned opposite to each other across the first access flap 4. The first fixed edge 41 is hingedly connected to the posterior-bracing portion 12 so the wearer or user cannot completely separate the first access flap 4 from the diaper body 1. The first free edge 42 is attached onto the crotch-bracing portion 13. The first free edge 42 is positioned adjacent to the anterior-bracing portion 11. In the preferred embodiment of the present invention, the first free edge 42 may be attached onto the crotch-bracing portion 13 utilizing any fastener such as, but not limited to, a hook-and-loop fastener or snap-button fastener. The arrangement of the first free edge 42 and the first fixed edge 41 allows the user to open or close the first access flap 4 when accessing the plurality of wipes 2.

As can be seen in FIGS. 1, 2 and 4 and in the preferred embodiment of the present invention, the disposable bag 3 is positioned onto the posterior-bracing portion 12 and is coextensively positioned to the posterior-bracing portion 12 preventing the disposable bag 3 from being contaminated during the diaper-changing process. Furthermore, the positioning of the disposable bag 3 prevents the wearer from accessing the disposable bag 3 while wearing the present invention. Moreover, the second access flap 5 comprises a second fixed edge 51 and a second free edge 52. The second fixed edge 51 and the second free edge 52 are positioned opposite to each other across the second access flap 5. The second fixed edge 51 is hingedly connected to the posterior-bracing portion 12 so that the wearer or user cannot completely separate the second access flap 5 from the diaper body 1. The second free edge 52 is attached onto the posterior-bracing portion 12. In the preferred embodiment of the present invention, the second free edge 52 may be attached onto the posterior-bracing portion 12 utilizing any fastener such as, but not limited to, a hook-and-loop fastener or snap-button fastener. The arrangement of the first free edge 42 and the first fixed edge 41 allows the user open or close the first access flap 4 when accessing the disposable bag 3.

As can be seen in FIGS. 1 and 2 and in the preferred embodiment, the second access flap 5 may further comprise a primary peeling indicator 53. The primary peeling indicator 53 provides a means for the user to easily identify where to open or close the second access flap 5. The primary peeling indicator 53 is integrated into the posterior-bracing portion 12. The primary peeling indicator 53 is positioned adjacent to the second free edge 52. The arrangement of the primary peeling indicator 53 allows the user to visually differentiate the second fixed edge 51 from the second free edge 52.

As can be seen in FIGS. 1 and 2, the present invention may further comprise a plurality of diaper-changing accessory compartments 6. The plurality of diaper-changing accessory compartments 6 is used to store a variety of diaper-changing accessories. In an alternative embodiment of the present invention, the plurality of diaper-changing accessory compartments 6 is pre-packaged with the variety of diaper-changing accessories. The plurality of diaper-changing accessory compartments 6 is integrated into the posterior-bracing portion 12 and is positioned adjacent to the disposable bag 3. The arrangement of the plurality of diaper-changing accessory compartments 6 prevents the wearer of the present invention, primarily an infant, from accessing the diaper-changing accessories.

Each of the plurality of diaper-changing accessory compartments 6 comprises a compartment flap 61. The compartment flap 61 conceals each of the plurality of diaper-changing compartments therefore preventing the stored diaper-changing accessories from falling out. The compartment flap 61 comprises a compartment fixed edge 611 and a compartment free edge 612. The compartment fixed edge 611 and the compartment free edge 612 are positioned opposite to each other across the compartment flap 61. The compartment fixed edge 611 is hingedly connected to the posterior-bracing portion 12 so that the wearer or user cannot completely separate the compartment flap 61 from the diaper body 1. The compartment free edge 612 is attached onto the posterior-bracing portion 12. In the preferred embodiment of the present invention, the compartment free edge 612 is attached onto the posterior-bracing portion 12 using, but not limited to, hook-and-loop fasteners. The arrangement of the compartment free edge 612 and the compartment fixed edge 611 allows the user to open or close the compartment flap 61 when accessing the diaper-changing accessories stored within each of the plurality of diaper-changing accessory compartments 6.

As can be seen in FIGS. 1 and 2 and in the preferred embodiment and similar to the second access flap 5, the compartment flap 61 may further comprise a secondary peeling indicator 613. The secondary peeling indicator 613 provides a means for the user to easily open or close the compartment flap 61. The secondary peeling indicator 613 is integrated into the posterior-bracing portion 12. The secondary peeling indicator 613 is positioned adjacent to the compartment free edge 612. The arrangement of the secondary peeling indicator 613 allows the user to visually differentiate the compartment fixed edge 611 from the compartment free edge 612.

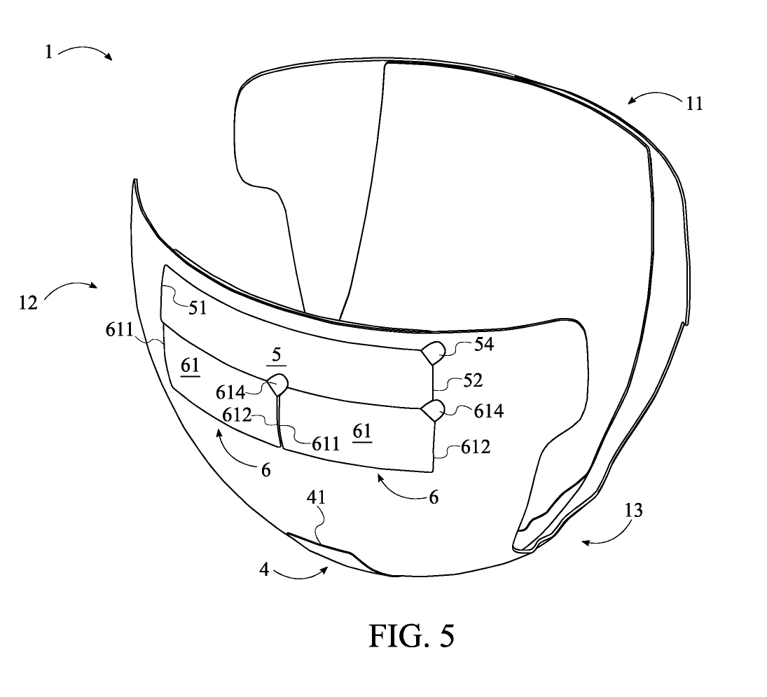

With reference to FIG. 5 and in another embodiment of the present invention, the second access flap 5 may further comprise a primary pull tab 54. The primary pull tab 54 provides a means for the user to easily open or close the second access flap 5. The primary pull tab 54 is connected adjacent to the second free edge 52. Similar, to the primary peeling indicator 53, the arrangement of the primary pull tab 54 allows the user to visually distinguish the second fixed edge 51 from the second free edge 52. The compartment flap 61 may further comprise a secondary pull tab 614. The secondary pull tab 614 provides a means for the user to easily open or close the second access flap 5. The secondary pull tab 614 is connected adjacent to the compartment free edge 612. Similar, to the secondary peeling indicator 613, the arrangement of the secondary pull tab 614 allows the user to visually distinguish the compartment fixed edge 611 from the compartment free edge 612.

Although the invention has been explained in relation to its preferred embodiment, it is to be understood that many other possible modifications and variations can be made without departing from the spirit and scope of the invention as hereinafter claimed.

What is claimed is:

1. A travel diaper comprises:
a diaper body;
a plurality of wipes;
a disposable bag;
a first access flap;
a second access flap;
the diaper body comprises an anterior-bracing portion, a posterior-bracing portion, and a crotch-bracing portion;
the disposable bag and the plurality of wipes being externally positioned to the diaper body;
the plurality of wipes being removably attached to the diaper body by the first access flap; and
the disposable bag being removably attached to the diaper body by the second access flap.

2. The travel diaper as claimed in claim 1 comprises:
the plurality of wipes being positioned onto the crotch-bracing portion and posterior bracing portion; and
the plurality of wipes being positioned adjacent to the anterior-bracing portion.

3. The travel diaper as claimed in claim 1 comprises:
the first access flap comprises a first fixed edge and a first free edge;
the first fixed edge and the first free edge being positioned opposite to each other across the first access flap;
the first fixed edge being hingedly connected to the posterior-bracing portion;
the first free edge being attached onto the crotch-bracing portion; and
the first free edge being positioned adjacent to the anterior-bracing portion.

4. The travel diaper as claimed in claim 1 comprises:
the disposable bag being positioned onto the posterior-bracing portion; and
the disposable bag being coextensively positioned to the posterior-bracing portion.

5. The travel diaper as claimed in claim 1 comprises:
the second access flap comprises a second fixed edge and a second free edge;
the second fixed edge and the second free edge being positioned opposite to each other across the second access flap;
the second fixed edge being hingedly connected to the posterior-bracing portion; and
the second free edge being attached onto the posterior-bracing portion.

6. The travel diaper as claimed in claim 5 comprises:
the second access flap further comprises a primary peeling indicator;
the primary peeling indicator being integrated into the posterior-bracing portion; and
the primary peeling indicator being positioned adjacent to the second free edge.

7. The travel diaper as claimed in claim 5 comprises:
the second access flap further comprises a primary pull tab; and
the primary pull tab being connected adjacent to the second free end.

8. The travel diaper as claimed in claim 1 comprises:
a plurality of diaper-changing accessory compartments;
the plurality of diaper-changing accessory compartments being integrated into the posterior-bracing portion; and
the plurality of diaper-changing accessory compartments being positioned adjacent to the disposable bag.

9. The travel diaper as claimed in claim 8 comprises:
each of the plurality of diaper-changing accessory compartments comprises a compartment flap;
the compartment flap comprises a compartment fixed edge and a compartment free edge;
the compartment fixed edge and the compartment free edge being positioned opposite to each other across the compartment flap;
the compartment fixed edge being hingedly connected to the posterior-bracing portion; and
the compartment free edge being attached onto the posterior-bracing portion.

10. The travel diaper as claimed in claim 9 comprises:
the compartment flap further comprises a secondary peeling indicator;
the secondary peeling indicator being integrated into the posterior-bracing portion; and
the secondary peeling indicator being positioned adjacent to the compartment free edge.

11. The travel diaper as claimed in claim 9 comprises:
the compartment flap further comprises a secondary pull tab; and
the secondary pull tab being connected adjacent to the compartment free end.

12. A travel diaper comprises:
a diaper body;
a plurality of wipes;
a disposable bag;
a first access flap;
a second access flap;
a plurality of diaper-changing accessory compartments;
the diaper body comprises an anterior-bracing portion, a posterior-bracing portion, and a crotch-bracing portion;
the disposable bag and the plurality of wipes being externally positioned to the diaper body;
the plurality of wipes being removably attached to the diaper body by the first access flap;
the disposable bag being removably attached to the diaper body by the second access flap;
the plurality of diaper-changing accessory compartments being integrated into the posterior-bracing portion; and
the plurality of diaper-changing accessory compartments being positioned adjacent to the disposable bag.

13. The travel diaper as claimed in claim 12 comprises:
the plurality of wipes being positioned onto the crotch-bracing portion and posterior bracing portion;
the plurality of wipes being positioned adjacent to the anterior-bracing portion;
the disposable bag being positioned onto the posterior-bracing portion; and
the disposable bag being coextensively positioned to the posterior-bracing portion.

14. The travel diaper as claimed in claim 12 comprises:
the first access flap comprises a first fixed edge and a first free edge;
the first fixed edge and the first free edge being positioned opposite to each other across the first access flap;
the first fixed edge being hingedly connected to the posterior-bracing portion;
the first free edge being attached onto the crotch-bracing portion; and
the first free edge being positioned adjacent to the anterior-bracing portion.

15. The travel diaper as claimed in claim 12 comprises:
the second access flap comprises a second fixed edge and a second free edge;
the second fixed edge and the second free edge being positioned opposite to each other across the second access flap;
the second fixed edge being hingedly connected to the posterior-bracing portion; and
the second free edge being attached onto the posterior-bracing portion.

16. The travel diaper as claimed in claim 15 comprises:
the second access flap further comprises a primary peeling indicator;
the primary peeling indicator being integrated into the posterior-bracing portion; and
the primary peeling indicator being positioned adjacent to the second free edge.

17. The travel diaper as claimed in claim 15 comprises:
the second access flap further comprises a primary pull tab; and
the primary pull tab being connected adjacent to the second free end.

18. The travel diaper as claimed in claim 12 comprises:
each of the plurality of diaper-changing accessory compartments comprises a compartment flap;
the compartment flap comprises a compartment fixed edge and a compartment free edge;
the compartment fixed edge and the compartment free edge being positioned opposite to each other across the compartment flap;
the compartment fixed edge being hingedly connected to the posterior-bracing portion; and
the compartment free edge being attached onto the posterior-bracing portion.

19. The travel diaper as claimed in claim 18 comprises:
the compartment flap further comprises a secondary peeling indicator;
the secondary peeling indicator being integrated into the posterior-bracing portion; and
the secondary peeling indicator being positioned adjacent to the compartment free edge.

20. The travel diaper as claimed in claim 18 comprises:
the compartment flap further comprises a secondary pull tab; and
the secondary pull tab being connected adjacent to the compartment free end.
